(19) United States
(12) Patent Application Publication
(10) Pub. No.: US 2022/0184075 A1
(43) Pub. Date: Jun. 16, 2022

(54) PHARMACEUTICAL COMPOSITION CONTAINING HDAC6 INHIBITOR AS ACTIVE INGREDIENT FOR PREVENTION OR TREATMENT OF ITCHING

(71) Applicant: BNH RESEARCH CO.,LTD., Gyeonggi-do (KR)

(72) Inventors: Jun Ho Jang, Seoul (KR); Jeong Ah Yoon, Gyeonggi-do (KR)

(21) Appl. No.: 17/687,929

(22) Filed: Mar. 7, 2022

Related U.S. Application Data
(63) Continuation of application No. 16/961,302, filed on Jul. 10, 2020.

(30) Foreign Application Priority Data
Jan. 12, 2018 (KR) 10-2018-0004434

(51) Int. Cl. A61K 31/505; A61K 31/166; A61K 31/422; A61K 31/437

(52) U.S. Cl. CPC A61K 31/505; A61K 31/437; A61K 31/422; A61K 31/166

(57) ABSTRACT

The present invention relates to a pharmaceutical composition containing an HDAC6 inhibitor as an active ingredient for prevention or treatment of itching. The present invention provides a novel pharmaceutical composition containing, as an active ingredient, an inhibitor for inhibiting an HDAC6 enzyme, and thus can effectively inhibit and treat itching caused by trypsin, tryptase, histamine, antimycin A, chloroquine, or the like.

### TECHNICAL FIELD

The present invention relates to a pharmaceutical composition for preventing or treating itching, which includes a HDAC6 inhibitor as an active ingredient.

### BACKGROUND ART

Proteins are synthesized from intracellular DNA and then undergo transformation, and post-translational modification is a mechanism of rapidly modifying and regulating the function of a specific protein for cells to correspond to internal and external environmental changes. Phospholation for adding a phosphate group to a protein is known as the most representative post-translational protein modification, but acetylation has also been reported to play a critical role in the change in protein function. Acetylation or deacetylation is known to have various effects on the function of a protein by regulating a lysine residue in acetic acid, which is the basic unit constituting the protein, which is mediated by specific enzymes.

Histone deacetylases (HDACs), which are deacetylases that remove an acetate from a protein, were known to deacetylate a histone protein present in the cell nucleus at the time of its initial discovery, and were named HDACs. However, it has been reported that, besides histone, various proteins inside or outside the nucleus can also be acetylated.

For a human, 18 HDACs are known and divided into four classes depending on the homology with yeast HDAC. Here, 11 HDACs using zinc as a cofactor may be divided into three classes such as Class I (HDAC1, 2, 3, 8), Class II (Ha: HDAC4, 5, 7, 9; IIb: HDAC6, 10) and Class IV (HDAC11). In addition, seven HDACs of Class III (SIRT 1-7) use NAD⁺ as a cofactor instead of zinc (Bolden et al., Nat. Rev. Drug Discov. 2006, 5(9), 769-784).

Among HDACs, while belonging to the HDACs, FIDAC6 is the only one of the HDACs, located in the cytoplasm, not in the cell nucleus, and has the characteristic of also deacetylating a non-historic intracellular protein. The acetylation of non-histone proteins may induce rapidly and reversibly protein modification as a type of post-translational modification, similar to phosphorylation, and change its characteristics, thereby having a direct influence on the functions of these proteins.

Recently, drugs for inhibiting HDAC6 have attracted attention in treatment of various diseases, and are used to treat various diseases such as neurodegenerative diseases, for example, Parkinson's disease, Alzheimer's diseases, and Huntington's disease, cancer, cardiovascular diseases, inflammation, and depression. For example, a composition for preventing or treating myositis, which includes a HDAC6 inhibitor, is disclosed in Korean Patent No. 10-1723867.

The HDAC6 inhibitor has recently been reported to be effective in treatment of chemotherapy-induced neuropathic pain, but its role for itching which is very close to pain and its mechanism, has not been reported so far.

Itching or pruritus is an unpleasant sensation that causes an urge to scratch and the most common symptom of patients with dermal diseases. Most itching-related conventional studies specified that histamine causes itching, tried to treat itching using an anti-histamine, but it was reported that the efficiency of such an anti-histamine is very limited in clinical trials.

Recently, proteinase-activated receptors (PARs) are attracting much attention in relation to itching. Particularly, while a PAR2 receptor is widely distributed in peripheral sensory nerves and skin cells and has been known to contribute to pain through sensitization of peripheral nerves, recently, it has been reported to be significantly involved even in itching. It has been reported that a selective activator of the PAR2 receptor, trypsin, induces severe itching when injected into a normal skin layer, and the expression of PAR2 receptor and an intrinsic activator of the PAR2 receptor, tryptase, significantly increase in the skin of an atopic dermatitis patient with intolerable itching.

The inventors newly found that the inhibition of HDAC6 can alleviate itching involved in the expression of histamine or the PAR2 receptor, and the present invention was completed.

### DISCLOSURE

### Technical Problem

The present invention is directed to providing a pharmaceutical composition for preventing or treating itching, which includes a HDAC6 inhibitor as an active ingredient.

To attain the above object, the present invention provides a pharmaceutical composition for preventing or treating itching, which includes a HDAC6 inhibitor as an active ingredient.

In the present invention, the HDAC6 inhibitor may be one or more selected from the group consisting of tubastatin A, tubacin, M344, ACY-63, ACY-216, ACY-241 (Citarinostat), ACY-251, ACY-257, ACY-738, ACY-775, ACY-1215 (Ricolinostat), ISOX, ST-3-06, ST-2-92, nexturastat A, nexturastat B, HPOB, CAY10603, BRD9757, TCS HDAC6 20b, vorinostat (SAHA), trichostatin A (TSA) and apicidin.

In the present invention, the itching may be induced by one or more selected from the group consisting of trypsin, tryptase, histamine, antimycin A, chloroquine, β-alanine, endothelin, serotonin, poly(I:C), imiquimod, thymic stromal lymphopoietin (TSLP), allyl isothiocyanate, cinnamaldehyde and capsaicin.

### Advantageous Effects

As described above, the present invention provides a novel pharmaceutical composition containing an inhibitor for inhibiting a HDAC6 enzyme as an active ingredient, thereby effectively inhibiting and treating itching induced by trypsin, tryptase, histamine, antimycin A, chloroquine, or the like.

### DESCRIPTION OF DRAWINGS

FIG. 1 is a scratching behavior graph when trypsin, or trypsin and tubastatin A are injected into a normal mouse.

FIG. 2 is a scratching behavior graph when trypsin, or trypsin and M344 are injected into a normal mouse.

### MODES OF THE INVENTION

Hereinafter, particular embodiments of the present invention will be described in further detail. Unless defined otherwise, technical and scientific terms used herein have meanings ordinarily understood by those of ordinary skill in the art to which the present invention belongs. Generally, the nomenclature used herein is well known and commonly used in the art.

The present invention relates to a pharmaceutical composition for preventing or treating itching, which includes a HDAC6 inhibitor as an active ingredient.

Most itching-related conventional studies specified that histamine causes itching and tried to treat itching using an antihistamine. However, the inventors studied a method for treating itching induced by various chemicals, including itching caused by an increased selective activator of a PAR2 receptor, that is, trypsin, without limitation to histamine-induced itching, and found that itching can be treated by inhibiting HDAC6.

A HDAC6 inhibitor included in the pharmaceutical composition of the present invention as an active ingredient is not particularly limited as long as itching can be prevented or treated by inhibiting a HDAC6 enzyme, and a selective HDAC6 inhibitor is preferably used, but a non-selective HDAC6 inhibitor can also be used as long as it does not cause other side effects.

For example, as a HDAC6 inhibitor that can be used in the present invention, tubastatin A, tubacin, M344, ACY-63, ACY-216, ACY-241 (Citarinostat), ACY-251, ACY-257, ACY-738, ACY-775, ACY-1215 (Ricolinostat), ISOX, ST-3-06, ST-2-92, nexturastat A, nexturastat B, HPUB, CAY10603, BRD9757, TCS HDAC6 20b, vorinostat (SAHA), trichostatin A (TSA) or apicidin may be used.

Representative HDAC6 inhibitors that can be used in the present invention are shown in Table 1 below.

TABLE 1

| | | IC₅₀ | |
|---|---|---|---|
| HDAC6 inhibitor | Selectivity | (nM) | Reference |
| Tubastatin A | Very high | 15 | WO2011-011186 |
| | | | International |
| | | | Immunopharmacology, |
| | | | Vol. 16, Issue 1, May 2013, |
| | | | Pages 72-78 |
| Tubacin | Very high | 4 | J. Am. Chem. Soc., 2003, 125, |
| | | | 5586-5587. |
| | | | J. Med. Chem., 2006, 49, |
| | | | 4809-4812 |
| M344 | High | 88 | PLoS ONE7(11): e48832. |
| ACY-1215 | High | 5 | Blood, 2012, 119, 2579-2589. |
| (Ricolinostat) | | | J. Med. Chem., 2014, 57 (19), |
| | | | pp 8026-8034 |
| ACY-241 | High | 4 | Blood, 2015 126: 3040 |
| (Citarinostat) | | | |
| ACY-738 | Very high | 1.7 | Neuropsychopharmacology |
| | | | (2014) 39, 389-400 |
| ACY-775 | Very high | 7.5 | Neuropsychopharmacology |
| | | | (2014) 39, 389-400 |
| HPOB | High | 56 | Mini Reviews in Medicinal |
| | | | Chemistry, Vol. 16, No. 14, |
| | | | September 2016, pp. |
| | | | 1175-1184(10) |
| CAY10603 | Very high | 0.002 | JOURNAL OF THORACIC |
| | | | ONCOLOGY, Vol. 10, No. 9, |
| | | | pp. S313-S313 |
| Nexturastat A | Very high | 5 | J. Med. Chem. 2012, 55, 9891- |
| | | | 9899. |
| BRD9757 | Very high | 30 | J. Med. Chem. 2013, 56, 1772- |
| | | | 1776 |

In Table 1, IC-₅₀ means a concentration required for an inhibitor inhibiting an inhibition target by 50%, and as the IC₅₀ is lower, the inhibition target can be inhibited by 50% with a smaller concentration of the inhibitor.

In one embodiment of the present invention, it was confirmed that, when tubastatin A and M344 as HDAC6 inhibitors are injected into mice, scratching behavior induced by trypsin can be inhibited. This means that the inhibition of HDAC6 is effective in treatment of itching, and a HDAC6 inhibitor which is not directly tested is also predicted to exhibit an effect which is similar to or same as the above effect.

Itching that can be treated by a pharmaceutical composition for preventing or treating itching, which includes the HDAC6 inhibitor of the present invention, may be itching induced by a chemical such as trypsin, tryptase, histamine, antimycin A, chloroquine, β-alanine, endothelin, serotonin, poly (I:C), imiquimod, thymic stromal lymphopoietin (TSLP), allyl isothiocyanate, cinnamaidehyde or capsaicin.

In one embodiment of the present invention, it was confirmed through a behavioral experiment that a selective activator of the PAR2 receptor, trypsin, induces the scratching behavior of a healthy mouse, and the scratching behavior can be inhibited by the HDAC6 inhibitor.

The pharmaceutical composition according to the present invention may include a pharmaceutically acceptable carrier. Here, the pharmaceutically acceptable carrier is conventionally used in drug preparation, and includes lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, and mineral oil, but the present invention is not limited thereto.

As well as the above components, the pharmaceutical composition according to the present invention may further include a lubricant, a wetting agent, a sweetening agent, a flavoring agent, an emulsifier, a suspending agent, or a preservative. Suitable pharmaceutically acceptable carriers and their preparations may be prepared according to each ingredient using a method disclosed in the Remington's Pharmaceutical Sciences (19th ed., 1995).

The pharmaceutical composition of the present invention may be administered either orally or parenterally, and parenteral administration includes intravenous injection, subcutaneous injection, intramuscular injection, intraperitoneal injection, and transdermal administration.

The pharmaceutical composition of the present invention is administered at a pharmaceutically effective amount. The “pharmaceutically effective amount” used herein refers to an amount sufficient for treating a disease at a reasonable benefit/risk ratio applicable for medical treatment, and an effective dosage may be determined by parameters including a type of a patient's disease, severity, drug activity, sensitivity to a drug, administration time, an administration route and an excretion rate, the duration of treatment and drugs simultaneously used, and other parameters well known in the medical field. The pharmaceutical composition of the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without a side effect, and such a dose may be easily determined by one of ordinary skill in the art.

Specifically, the effective amount of the composition of the present invention may vary according to a patient's age, sex or body weight, and may be generally administered at 0.001 to 150 mg, and preferably, 0.01 to 100 mg, per kg of body weight daily or every other day, or divided into one or three daily administrations. However, the effective amount may vary depending on an administration route, sex, a body weight or an age, and therefore, the scope of the present invention is not limited by the dose in any way.

The pharmaceutical composition of the present application may be prepared by a unit dose packaging or multi-dose packaging after being prepared in a conventional dosage form using a pharmaceutically acceptable carrier and/or excipient according to a method capable of being performed by those of ordinary skill in the art. Here, the dosage form may be a solution, suspension or emulsion in an oil or aqueous medium, an extract, a powder, a granule, a tablet, or a capsule, and may further include a dispersing agent or a stabilizer.

The active ingredient used in the composition of the present invention is not only the HDAC6 inhibitor, but also a pharmaceutically acceptable salt, hydrate, solvate or prodrug thereof.

The term “pharmaceutically acceptable salt” used herein denotes a salt of the HDAC6 inhibitor having a desired pharmacological effect, that is, a HDAC6 inhibitory effect. The salt may be formed using an inorganic acid such as hydrochlorides, hydrobromides or hydroiodides, or an organic acid such as acetates, adipates, alginates, aspartates, benzoates, benzosulfonates, p-toluenesulfonates, bisulfates, sulfamates, sulfates, naphthylates, butyrates, citrates, comphorates, camposulfonates, cyclopentanepropionates, digluconates, dodecyl sulfates, ethanesulfonates, fumarates, glucoheptanoates, glycerophosphates, hemisulfates, heptanoates, hexanoates, 2-hydroxyethanesulfates, lactates, maleates, methanesulfonates, 2-naphthalenesulfonates, nicotinates, oxalates, tosylates or undecanoates.

The term “pharmaceutically acceptable hydrate” used herein denotes a hydrate of the HDAC6 inhibitor having a desired pharmacological effect.

The term “pharmaceutically acceptable solvate” used herein denotes a solvate of the HDAC6 inhibitor having a desired pharmacological effect. The hydrate and solvate may also be prepared using the above-described salts.

The term “pharmaceutically acceptable prodrug” used herein denotes a derivative of the HDAC6 inhibitor that should be converted in the body before the pharmacological effect of the HDAC6 inhibitor is exhibited. The prodrug is prepared to prolong the duration of action and reduce side effects to improve chemical stability, patient acceptability, bioavailability, organ selectivity or ease of preparation. The prodrug of the present invention may be easily prepared according to a conventional method in the art (e.g., Burger's Medicinal Chemistry and Drug Chemistry, 5th ed., 1:172-178 and 949-982(1995)) using the HDAC6 inhibitor.

### EXAMPLES

Hereinafter, the present invention will be described in more detail with reference to examples. The examples are merely provided to more fully describe the present invention, and it will be obvious to those of ordinary skill in the art that the scope of the present invention is not limited to the following examples.

Experimental Preparation and Methods

Male C57BL/6 mice (6-week-old) were divided into groups of 3 to 5 animals, and isolated under a 12-hour light/dark cycle while being allowed to freely access water and food.

All chemicals used in this example are commercially available from Sigma-Aldrich.

Trypsin is diluted in phosphate buffered saline (PBS) to have the working concentration.

A 10 mM stock solution was prepared by dissolving each of selective HDAC6 inhibitors, such as tubastatin A HCl and M344, in dimethyl sulfoxide (DMSO).

All drug-containing stock solutions were stored in a freezer at −20 □, dissolved immediately prior to the behavioral experiment, and diluted with PBS to the final working concentration.

For behavioral experiments, the right cheek of all mice was shaved, and the drug was injected after the acclimation period in an observation chamber for 2 days (2 hours/day) prior to the experiment date and 1 hour on the day of the experiment.

The mice were subdued by being gently grabbed by a hand without anesthesia for drug treatment. All drugs were injected into the skin layer of the shaved right cheek at 20 μL using a 31-G insulin syringe.

After returning to the observation chamber, all mice were videotaped for 30 minutes immediately after drug injection to evaluate itching behavior. For the behavioral experiment, the action of scratching the drug-injected site with a hind leg, and then placing the leg on the ground again or licking the site with a mouth was counted as 1 bout. All behavioral experiments were evaluated by testers in a blinded drug test.

Statistical analysis for the experiments was performed using an unpaired t-test, and P<0.05 or P<0.01 was considered statistically significant (*: P<0.05; **: P<0.01). Data was expressed as mean±standard error (SEM).

### Example 1

### Experiment for Treating Itching by Tubastatin A

Scratching behavior after normal mice were injected with 200 μg of trypsin and 7.4 μg of tubastatin A HCl was measured for 30 minutes, and compared with that of a group injected only with 200 μg of trypsin.

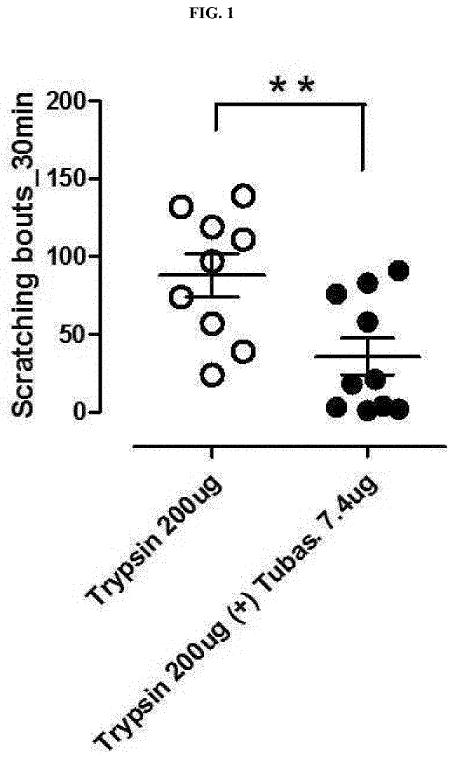

As confirmed in FIG. 1, the trypsin-injected mice showed approximately 100 scratching bouts for 30 minutes, confirming that itching was induced by trypsin.

In addition, the mice injected with tubastatin A along with trypsin to inhibit HDAC6 showed a statistically significant smaller number of scratching bouts, compared with the only trypsin-injected mice without tubastatin A injection.

That is, it was experimentally confirmed that a selective HDAC6 inhibitor, tubastatin A, can significantly alleviate itching induced by trypsin.

### Example 2

### Experiment for Treating Itching by M344

A behavioral experiment for itching was performed in the same manner as described in Example 1, except that 61.4 ng of M344, instead of tubastatin A, was injected.

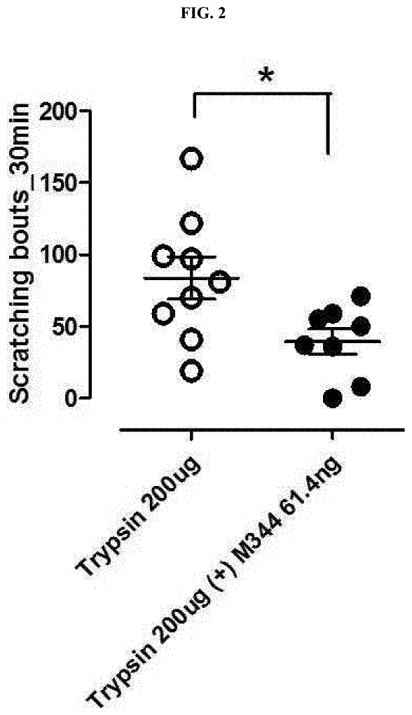

As confirmed in FIG. 2, mice injected with M344 along with trypsin to inhibit HDAC6 showed a statistically significant smaller number of scratching bouts, compared with the only trypsin-injected mice without M344 injection.

That is, it was experimentally confirmed that a selective HDAC6 inhibitor, M344, can significantly alleviate itching induced by trypsin.

As above, although some embodiments of the present invention have been described, the present invention is not limited only to the above-described embodiments, but may also be implemented by alterations and modifications without departing from the gist of the present invention. It should be understood that the alterations and modifications also belong to the technical spirit of the present invention.

1-5. (canceled)

6. A method for alleviating or treating itching, the method comprising administering a composition comprising a HDAC6 inhibitor as an active ingredient, to a subject in need thereof.

7. The method of claim 6, wherein the HDAC6 inhibitor is one or more selected from the group consisting of tubastatin A, tubacin, M344, ACY-63, ACY-216, ACY-241 (Citarinostat), ACY-251, ACY-257, ACY-738, ACY-775, ACY-1215 (Ricolinostat), ISOX, ST-3-06, ST-2-92, nexturastat A, nexturastat B, IIPOB, CAY10603, BRD9757, TCS HDAC6 20b, vorinostat (SAHA), trichostatin A, tubacin, M344, ACY-241 and ACY-1215.

8. The method of claim 6, wherein the HDAC6 inhibitor is one or more selected form the group consisting of tubastatin A, tubacin, M344, ACY-241 and ACY-1215.

9. The method of claim 6, wherein the itching is induced by one or more selected from the group consisting of trypsin, tryptase, histamine, antimycin A, chloroquine, β-alanine, endothelin, serotonin, poly(I:C), imiquimod, thymic stromal lymphopoietin (TSLP), allyl isothiocyanate, cinnamaldehyde and capsaicin.

10. The method of claim 6, wherein the itching is induced by one or more selected from the group consisting of trypsin, tryptase, histamine, antimycin A and chloroquine.

6. The method of claim 1, wherein the composition reduces scratching.
